(19) United States
(12) Patent Application Publication
(10) Pub. No.: US 2022/0369974 A1
(43) Pub. Date: Nov. 24, 2022

(54) Mental Health Tracking

(71) Applicant: AT&T Intellectual Property I, L.P., Atlanta, GA (US)

(72) Inventor: Ajay Singh, Hillsborough, NJ (US)

(73) Assignee: AT&T Intellectual Property I, L.P., Atlanta, GA (US)

(21) Appl. No.: 17/323,060

(22) Filed: May 18, 2021

(51) Int. Cl. A61B 5/16; A61B 5/375; A61B 5/00

(52) U.S. Cl. CPC A61B 5/165; A61B 5/375; A61B 5/743

(57) ABSTRACT

Concepts and technologies disclosed herein are directed to mental health tracking. According to one aspect of the concepts and technologies disclosed herein, a system can obtain electroencephalography (“EEG”) data associated with a user. The EEG data can be obtained from an EEG device worn by the user. The EEG data can include real-time EEG data and/or historical EEG data. The system can obtain calendar data associated with the user. The calendar data can identify a calendar event such as a business meeting. The calendar data can be associated with a calendar of the user and/or a shared calendar of an entity other than the user. The system can map the calendar data to the EEG data. The system can then provide feedback to the user. The feedback can provide insight regarding a stress level of the user in association with the calendar event.

### BACKGROUND

Electroencephalography (“EEG”) is the act of measuring and recording electrical activity on the skin of the scalp to record brainwaves caused by neurons firing in the brain. EEG is used in the medical field to evaluate patients that experience symptoms of conditions such as epilepsy, Alzheimer's disease, tumors, stroke, and sleep disorders (e.g., narcolepsy) to aid in diagnosis. In recent years, EEG devices have become available at relatively low cost, and as a result, the application of this technology has expanded beyond the medical field to other fields, such as entertainment (e.g., video games), education, research, and toys.

### SUMMARY

Concepts and technologies disclosed herein are directed to mental health tracking. According to one aspect of the concepts and technologies disclosed herein, a system can obtain EEG data associated with a user. The EEG data can be obtained from an EEG device worn by the user. The EEG data can include real-time EEG data and/or historical EEG data. The system can obtain calendar data associated with the user. The calendar data can identify a calendar event such as a business meeting. The calendar data can be associated with a calendar of the user and/or a shared calendar of an entity other than the user. The system can map the calendar data to the EEG data. The system can then provide feedback to the user. The feedback can provide insight regarding a stress level of the user in association with the calendar event.

In some embodiments, the system also can obtain other data associated with the user. The other data can be or can include data from one or more other applications executed by the system. The other data can include health tracking data, social media data, communications data, combinations thereof, and/or the like.

In some embodiments, the system can utilize machine learning to generate the feedback. In these embodiments, the feedback can include one or more recommendations to the user. For example, a recommendation may be to change a time of the calendar event to earlier or later in the day when the user is less prone to stress and/or for the user to avoid caffeine prior to the calendar event.

In some embodiments, the feedback can include a visualization of the stress level of the user in association with the calendar event. In these embodiments, the system can apply various filters to allow the user to isolate specific types of data. For example, a filter may be applied to certain types of calendar events (e.g., business meetings) or based on other factors (e.g., a spouse working from home with the user and/or the user's children schooling from home).

It should be appreciated that the above-described subject matter may be implemented as a computer-controlled apparatus, a computer process, a computing system, or as an article of manufacture such as a computer-readable storage medium. These and various other features will be apparent from a reading of the following Detailed Description and a review of the associated drawings.

This Summary is provided to introduce a selection of concepts in a simplified form that are further described below in the Detailed Description. This Summary is not intended to identify key features or essential features of the claimed subject matter, nor is it intended that this Summary be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 is a block diagram illustrating an operating environment in which aspects of the concepts and technologies disclosed herein can be implemented.

FIG. 2 is a flow diagram illustrating aspects of a method for mental health tracking, according to an illustrative embodiment.

FIGS. 3-5 are diagrams illustrating various data visualizations in accordance with embodiments disclosed herein.

FIG. 6 is a block diagram illustrating an example computer system capable of implementing aspects of the embodiments presented herein.

FIG. 7 is a block diagram illustrating an example mobile device capable of implementing aspects of the embodiments disclosed herein.

FIG. 8 is a block diagram illustrating an example virtualized cloud architecture and components thereof capable of implementing aspects of the embodiments presented herein.

FIG. 9 is a diagram illustrating a machine learning system, according to an illustrative embodiment.

FIG. 10 is a diagram illustrating a network, according to an illustrative embodiment.

### DETAILED DESCRIPTION

While the subject matter described herein may be presented, at times, in the general context of program modules that execute in conjunction with the execution of an operating system and application programs on a computer system, those skilled in the art will recognize that other implementations may be performed in combination with other types of program modules. Generally, program modules include routines, programs, components, data structures, computer-executable instructions, and/or other types of structures that perform particular tasks or implement particular abstract data types. Moreover, those skilled in the art will appreciate that the subject matter described herein may be practiced with other computer systems, including hand-held devices, mobile devices, wireless devices, multiprocessor systems, distributed computing systems, microprocessor-based or programmable consumer electronics, minicomputers, mainframe computers, routers, switches, other computing devices described herein, and the like.

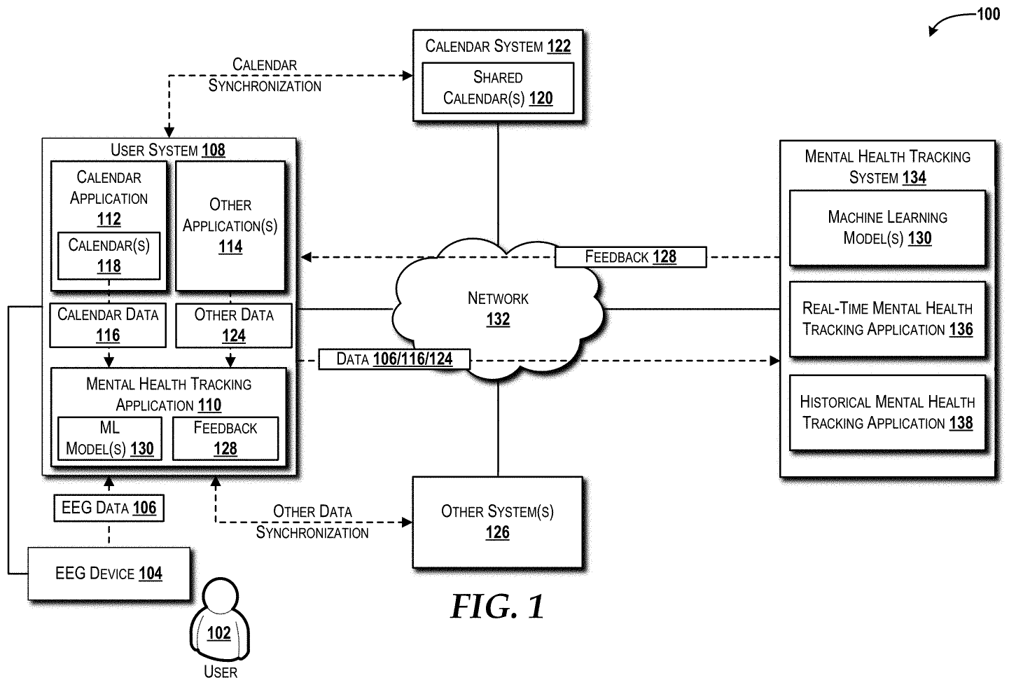

Referring now FIG. 1, a block diagram illustrating an operating environment 100 in which aspects of the concepts and technologies disclosed herein can be implemented will be described. The illustrated operating environment 100 includes a user 102 that can wear an EEG device 104 to capture EEG data 106 that is representative of their brain activity. The EEG data 106 can include raw EEG signals expressed in EEG frequencies and time stamps of when the EEG signals were captured. EEG signals within a frequency band from approximately 0.5 Hertz (“Hz”) to 2.75 Hz are referred to as EEG Delta waves and are associated with a deep meditative state of mind or when the user is in a deep sleep state. EEG signals within a frequency band from approximately 3.5 Hz to 6.75 Hz are referred to as EEG Theta waves and are associated with a meditative state of mind or when the user 102 is drowsy or in a sleep state. EEG signals within a frequency range from approximately 7.5 Hz to 9.25 Hz are referred to as low EEG Alpha waves and are associated with a relaxed, calm, and lucid state of mind in which the user 102 is able to think normally. EEG signals within a frequency range from approximately 10 Hz to 11.75 Hz are referred to as high EEG Alpha waves and are also associated with a relaxed, calm, and lucid state of mind in which the user 102 is able to think normally. High EEG Alpha waves may be representative of an increase in the user's 102 concentration. EEG signals within a frequency range from approximately 13 Hz to 16.75 Hz are referred to as low EEG Beta waves and are associated with the user 102 being conscious or in an awake, attentive, and alert state such as when the user 102 is focusing or thinking intently. EEG signals within a frequency range from approximately 18 Hz to 29.75 Hz are referred to as high EEG Beta waves and are also associated with the user 102 being conscious or in an awake, attentive, and alert state such as when the user 102 is focusing or thinking intently, although even more intensely than low EEG Beta waves. EEG signals within a frequency range from approximately 31 Hz to 39.75 Hz are referred to as low EEG Gamma waves and are associated with the user 102 being in a highly perceptive state of mind in which the user 102 is actively learning or solving problems. Mid EEG signals within a frequency range from approximately 41 Hz to 49.75 Hz are referred to as mid EEG Gamma waves and are also associated with the user 102 being in a highly perceptive state of mind in which the user 102 is actively learning or solving problems, although even more intensely than low EEG Gamma waves. It should be understood that the aforementioned frequency bands may extend beyond the stated minimum and maximum frequencies and still be categorized as Delta, Theta, Alpha, Beta, or Gamma waves. These frequency bands may also vary slightly among individuals. Accordingly, these frequency bands should be considered merely exemplary examples of various frequency bands and related EEG brainwaves.

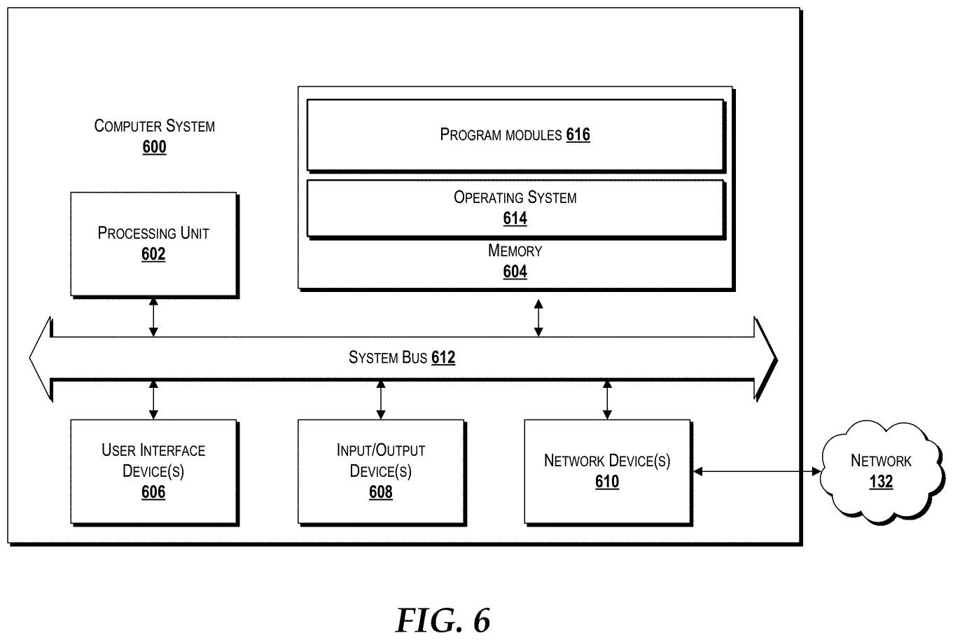

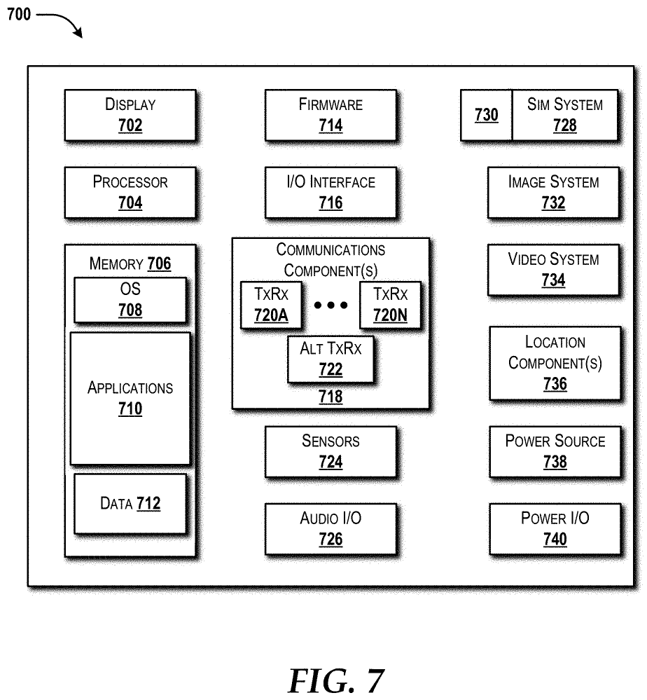

The EEG device 104 can be any off-the-shelf EEG device or custom EEG device. The EEG device 104 can include any number of electrodes used to attach to the user's 102 scalp. For example, the EEG device 104 can include a number of electrodes sufficient to cover the user's 102 scalp according to the International 10-20 system or other system for electrode placement that is currently used or will be developed in the future. The EEG device 104 can communicate with a user system 108 via BLUETOOTH, WI-FI, or other wireless technologies, and/or via wired technologies such as universal serial bus (“USB”) or Ethernet. The user system 108 may be embodied as a traditional desktop or laptop computer, a tablet, or a smartphone (examples shown in FIGS. 6 and 7). In some embodiments, the EEG device 104 is a consumer-grade device such as, but not limited to, MINDWAVE available from NEUROSKY, INC. In some embodiments, the EEG device 104 is a clinical-grade device such as, but not limited to, NEUROEEG available from MEMORYMD, INC. In some embodiments, the functionality of the EEG device 104 is built-in to the user system 108. EEG devices in general are well-known, and therefore additional details about the functionality of the EEG device 104 are not described herein.

The illustrated user system 108 can execute a mental health tracking application 110, a calendar application 112, and one or more other applications 114. The mental health tracking application 110 can obtain the EEG data 106 from the EEG device 104. The mental health tracking application 110 also can obtain calendar data 116 associated with one or more calendars 118 provided by the calendar application 112 and/or one or more shared calendars 120 (e.g., calendars shared with the user 102 by friends, family, co-workers, and/or other entities) synchronized between a calendar system 122 and the calendar application 112. The mental health tracking application 110 also can obtain other data 124 from the other application(s) 114 that can be synchronized with one or more other systems 126, such as one or more application servers. The other application(s) 114 can include health tracking applications used to track aspects of the user's 102 health, such as sleep, nutrition, hydration, activity, combinations thereof, and/or the like. The other application(s) 114 can include social media applications. The other application(s) 114 can include phone, text message, email, chat, other communication applications, combinations thereof, and/or the like. The other data 124 can include any data associated with the other application(s) 114, including data associated with the user 102 and/or other entities such as those associated with the shared calendar(s) 120.

The mental health tracking application 110 can map the calendar data 116 and/or the other data 124 to determine the impact of various factors, such as calendar events (e.g., business meetings, social gatherings, doctor's appointments, etc.) and/or other factors (e.g., user's sleep schedule, nutrition, hydration, activity, social interactions via social media, communications with others, etc.) on the brain activity of the user 102, and therefore the stress level experienced by the user 102 during the calendar event(s). For example, an increase in low-amplitude EEG Beta waves during the time leading up to or during an important business meeting (per the calendar data 116) may indicate that the user 102 is more anxious. A further increase in low-amplitude EEG Beta waves may indicate that the user 102 is adversely affected by an increase in caffeine consumption prior to that meeting. Conversely, EEG Alpha waves may be indicative of the user 102 being in a calm, relaxed state. Periods of stress and calm are both components of mental health tracking in accordance with the concepts and technologies disclosed herein. It should be understood that the mental health tracking application 110 may, in some implementations, be able to extrapolate additional insights into the mental health of the user 102, such as whether the user 102 appears to suffer from a mental disorder such as depression or social anxiety.

The mental health tracking application 110 can provide feedback 128 to the user 102 about the impact of various factors, such as those described above, on the user's 102 brain activity. The feedback 128 can be real-time feedback. For example, the user 102 can wear the EEG device 104 during a calendar event and receive the feedback 128 in real-time, such as in the form of one or more visualizations, which can include graphs (e.g., bar graphs, pie graphs, line graphs, etc.), text descriptions, reports, or some other form of visual feedback that can inform the user 102 of their current state of mind during the calendar event. Audio and video feedback are also contemplated. The state of mind may indicate that the user 102 is rational, anxious, depressed, angry, fearful, rebellious, stressed, calm, or some combination thereof. The feedback 128 can alternatively or additionally include historical feedback generated over time. The historical feedback can be in the form of a visualization, graph, text description, report, audio, video, or some other form of feedback that can inform the user 102 of their state of mind over time during their day-to-day activities. The historical feedback can help the user 102 identify trends such as when the user 102 is calmest or most stressed. In some embodiments, the mental health tracking application 110 can utilize one or more machine learning models 130 to identify trends in the user's 102 stress and/or calm levels. Moreover, the machine learning model(s) 130 can be used to identify and make recommendations for changes to the user's 102 schedule (e.g., change a meeting time) and/or behavior (e.g., avoid caffeine before a meeting).

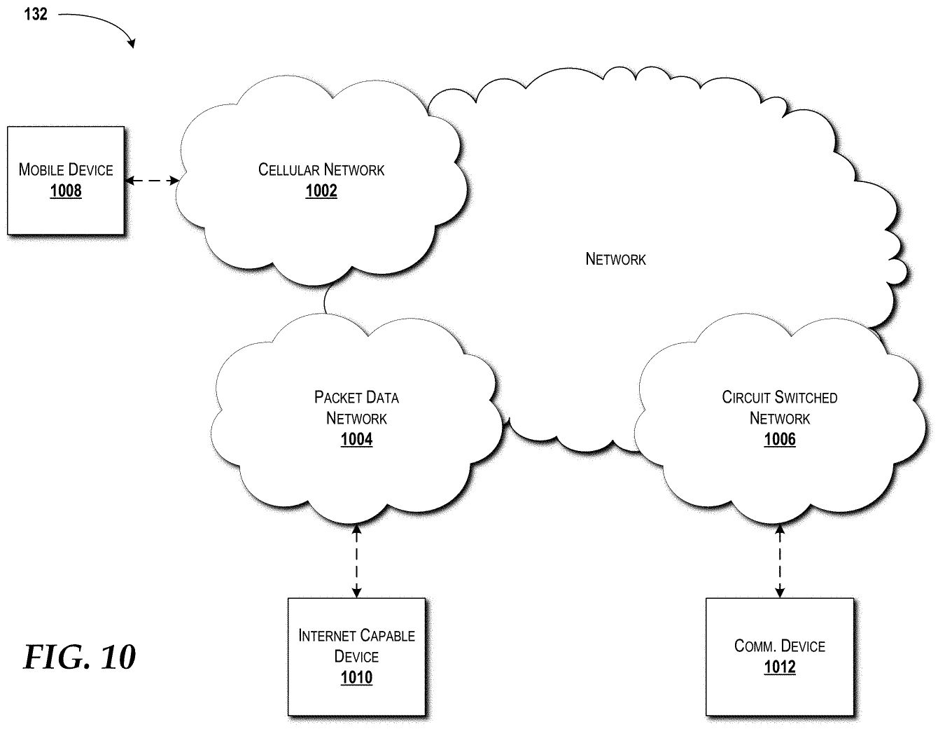

The user system 108 is shown operating in communication with a network 132 (shown in detail in FIG. 10). The network 132 can facilitate communications among the user system 108, the calendar system 122, the other system(s) 126, and a mental health tracking system 134. In some embodiments, the mental health tracking application 110 can operate as a client application to the mental health tracking system 134 or particular applications executed by the mental health tracking system 134. The mental health tracking system 134 can execute a real-time mental health tracking application 136 that can provide the feedback 128 in the form of real-time feedback to the mental health tracking application 110. The mental health tracking system 134 can execute a historical mental health tracking application 138 that can provide the feedback 128 in the form of historical feedback to the mental health tracking application 110. In some embodiments, the mental health tracking system 134 is used to offload computationally intensive operations, such as machine learning operations using the machine learning model(s) 130.

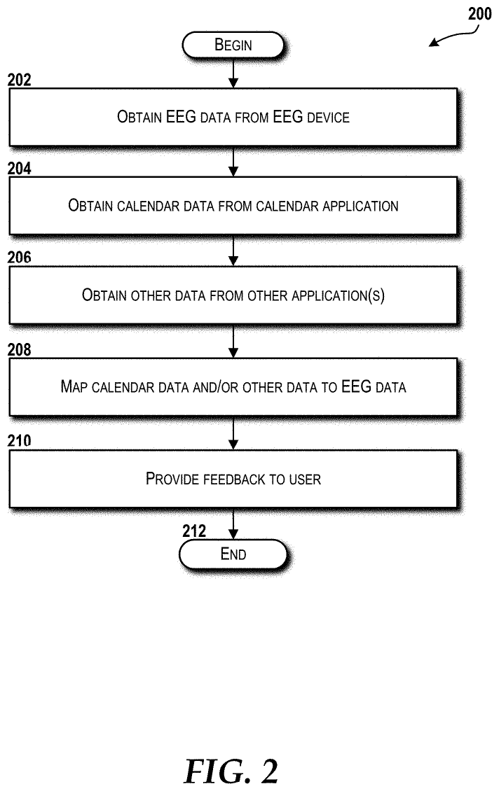

Turning now to FIG. 2, a method 200 for mental health tracking will be described, according to an illustrative embodiment. It should be understood that the operations of the methods disclosed herein are not necessarily presented in any particular order and that performance of some or all of the operations in an alternative order(s) is possible and is contemplated. The operations have been presented in the demonstrated order for ease of description and illustration. Operations may be added, omitted, and/or performed simultaneously, without departing from the scope of the concepts and technologies disclosed herein.

It also should be understood that the methods disclosed herein can be ended at any time and need not be performed in its entirety. Some or all operations of the methods, and/or substantially equivalent operations, can be performed by execution of computer-readable instructions included on a computer storage media, as defined herein. The term “computer-readable instructions,” and variants thereof, as used herein, is used expansively to include routines, applications, application modules, program modules, programs, components, data structures, algorithms, and the like. Computer-readable instructions can be implemented on various system configurations including single-processor or multiprocessor systems, minicomputers, mainframe computers, personal computers, hand-held computing devices, microprocessor-based, programmable consumer electronics, combinations thereof, and the like.

Thus, it should be appreciated that the logical operations described herein are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These states, operations, structural devices, acts, and modules may be implemented in software, in firmware, in special purpose digital logic, and any combination thereof. As used herein, the phrase “cause a processor to perform operations” and variants thereof is used to refer to causing a processor or multiple processors of one or more systems and/or one or more devices disclosed herein to perform one or more operations and/or causing the processor to direct other components of the computing system or device to perform one or more of the operations.

The method 200 will be described from the perspective of the user system 108 executing the mental health tracking application 110. In some embodiments, certain operations may be performed either locally by the user system 108, remotely via the mental health tracking system 134, or performed at least partially by the user system 108 and the mental health tracking system 134.

The method 200 begins and proceeds to operation 202. At operation 202, the user system 108, via execution of the mental health tracking application 110, can obtain the EEG data 106 from the EEG device 104. The EEG data 106 can contain raw EEG signals with different frequencies that fall within different frequency bands that are indicative of different types of brainwaves such as Delta, Theta, Alpha, Beta, and Gamma. The mental health tracking application 110 can determine from the frequency bands which EEG brainwaves are currently active in the user's 102 brain. Alternatively, the EEG data 106 may indicate the type of brainwaves measured and forego any reference to a specific frequency band. For example, if the EEG device 104 measures frequencies within 13 Hz and 29.75 Hz, the EEG data 106 can include an indication that EEG Beta waves have been measured without specificity regarding the actual frequencies measured. The granularity of the EEG data 106 (e.g., specific frequencies frequency bands or direct indication of brainwave type) can be selected based upon the needs of a given implementation of the concepts and technologies disclosed herein.

From operation 202, the method 200 proceeds to operation 204. At operation 204, the user system 108, via execution of the mental health tracking application 110, can obtain the calendar data 116 from the calendar application 112. The calendar data 116 can include dates and times of calendar events such as meetings or appointments in one or more of the calendars 118 associated with the user 102 and/or one or more of the shared calendars 120 associated with one or more friends, family, co-workers, and/or other entities that have given permission to the user 102, via the calendar application 112, to access their respective shared calendars 120.

From operation 204, the method 200 proceeds to operation 206. At operation 206, the user system 108, via execution of the mental health tracking application 110, can obtain the other data 124 from the other application(s) 114. The other application(s) 114 can include health tracking applications used to track aspects of the user's 102 health, such as sleep, nutrition, hydration, activity, combinations thereof, and/or the like. The other application(s) 114 can include social media applications. The other application(s) 114 can include phone, text message, email, chat, virtual meeting, other communication applications, combinations thereof, and/or the like. The other data 124 can include any data obtained from the other application(s) 114, some examples of which include sleep data, nutrition data, hydration data, activity data, phone call data, text message data, email data, chat data, social media data, combinations thereof, and the like.

From operation 206, the method 200 proceeds to operation 208. At operation 208, the user system 108, via execution of the mental health tracking application 110, can map the calendar data 116 and/or the other data 124 to the EEG data 106. In some embodiments, the date and time of the calendar data 116 can be mapped to the timestamp of the EEG data 106. For example, a calendar event on Jun. 1, 2021 at 1 PM EDT and scheduled to last 30 minutes can be mapped to the EEG data 106 obtained at or around 1 PM EDT and 30 minutes plus or minus to accommodate the entire meeting. The actual end time of the meeting can be obtained from the calendar application 112 or another application 114 embodied, for example, as a virtual meeting application. The other data 124 may be associated with metadata that can include a timestamp or other information indicative of when the other data 124 was captured.

From operation 208, the method 200 proceeds to operation 210. At operation 210, the user system 108, via execution of the mental health tracking application 110, can provide the feedback 128 to the user 102 about the impact of various factors, such as those described above, on the user's 102 brain activity. The feedback 128 can be real-time feedback. For example, the user 102 can wear the EEG device 104 during a calendar event and receive the feedback 128 in real-time, such as in the form of a visualization or some other form of feedback that can inform the user 102 of their current state of mind during the calendar event. The state of mind may indicate that the user 102 is rational, anxious, depressed, angry, fearful, rebellious, stressed, calm, or some combination thereof. The feedback 128 can alternatively or additionally include historical feedback, in which case the operations 202, 204, 206, 208 may be performed multiple times over a time period. The historical feedback also can be in the form of a visualization or some other form of feedback that can inform the user 102 of their state of mind over time during their day-to-day activities. The historical feedback can help the user 102 identify trends such as when the user 102 is calmest or most stressed. In some embodiments, the mental health tracking application 110 can utilize one or more machine learning models 130 to identify trends within the feedback 128. Moreover, the machine learning model(s) 130 can be used to identify and make recommendations for changes to the user's 102 schedule (e.g., change a meeting time) and/or behavior (e.g., avoid caffeine before a meeting) as part of the feedback 128. As mentioned above, the user system 108 can offload computationally intensive operations, such as machine learning operations to the mental health tracking system 134. Moreover, the mental health tracking system 134 can be used to store the EEG data 106, the calendar data 116, and the other data 124 obtained over time to reduce the storage burden on the user system 108.

From operation 210, the method 200 proceeds to operation 212. At operation 212, the method 200 can end.

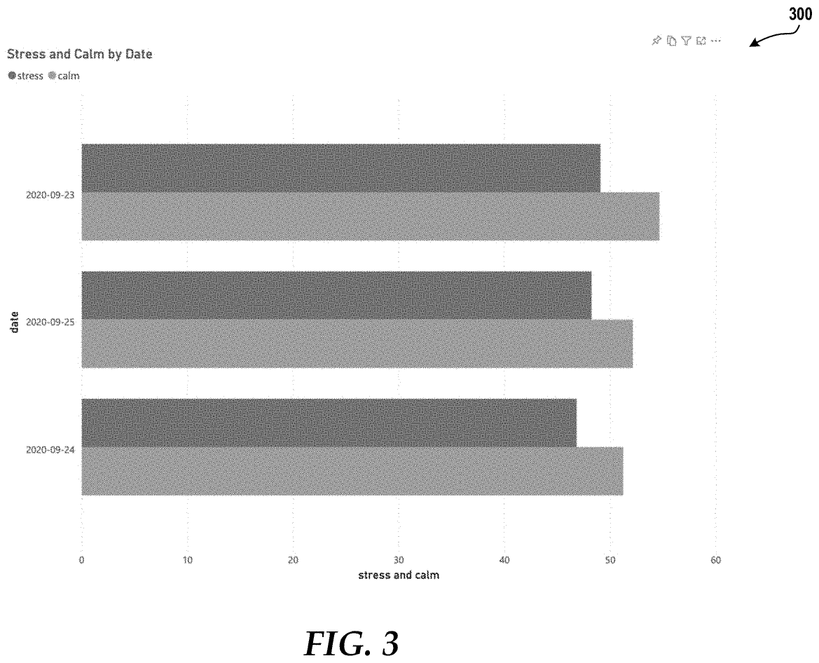

Turning now to FIGS. 3-5, various example data visualizations will be described in accordance with embodiments disclosed herein. FIG. 3 is a diagram illustrating a first example visualization 300 that shows levels of stress and calm experienced by the user 102 and categorized by date. The first example visualization 300 can be provided as the feedback 128 or a portion thereof. In the first example visualization 300, the date is represented on the y-axis and the level of stress/calm is represented on the x-axis. The level of stress/calm is represented as a normalized range of brainwaves from 0 to 100. Alternatively, raw frequency data or different normalization algorithms may be used. The first example visualization 300 does not provide any insight regarding what occurred on the dates (e.g., meetings, calls, or other factors). It should be understood, however, that the first example visualization 300 could be generated with additional granularity such as specific time periods during the dates listed and/or calendar events/other factor(s). Moreover, although the visualization 300 is shown as a bar graph, other graph types are contemplated, as are raw data reports such as in the form of a spreadsheet. The color, font, emphasis (e.g., bold or italics), and other visual aspects of the first example visualization 300 can be customized as needed or desired for a particular implementation. As such, the illustrated example should not be construed as being limiting in any way.

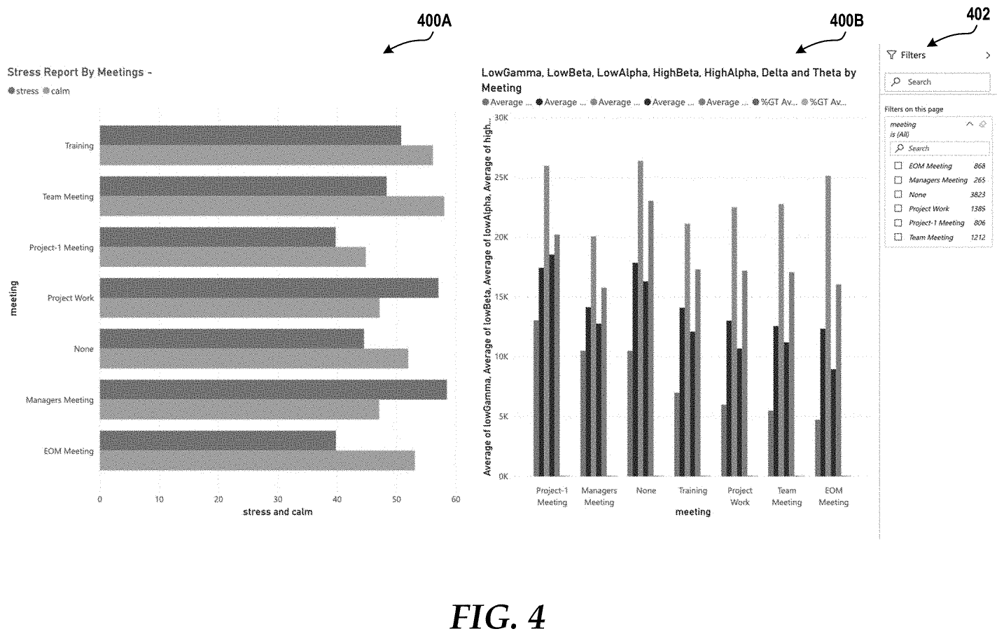

FIG. 4 is a diagram illustrating a second example visualization 400A that shows levels of stress and calm experienced by the user 102 during different meetings. The second example visualization 400A can be provided as the feedback 128 or a portion thereof. In the second example visualization 400A, the meeting type is represented on the y-axis and the level of stress/calm is represented on the x-axis. FIG. 4 also illustrates a third example visualization 400B that shows meeting types on the x-axis and the average EEG brainwaves captured, by the EEG device 104, from the user's 102 brain during each meeting type on the y-axis. The example visualizations 400A, 400B can be filtered using one or more filters 402 to isolate data associated with particular meeting types. Other filters are contemplated. Although the example visualizations 400A, 400B are shown as bar graphs, other graph types are contemplated, as are raw data reports such as in the form of a spreadsheet. The color, font, emphasis (e.g., bold or italics), and other visual aspects of the example visualizations 400A, 400B can be customized as needed or desired for a particular implementation. As such, the illustrated example should not be construed as being limiting in any way.

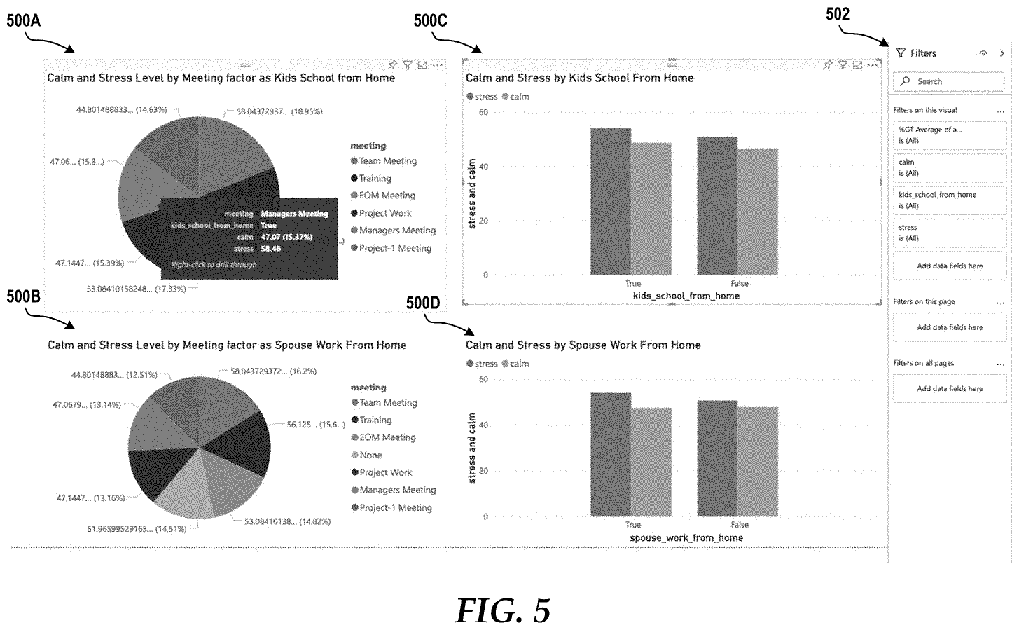

FIG. 5 is a diagram illustrating additional example visualizations 500A-500D will be described. The example visualizations 500A-500D are provided based on the assumption that the user 102 is working remotely from home. The example visualization 500A shows a pie graph of calm and stress levels experienced by the user 102 during meetings when the user's 102 kids attend school from home (i.e., remote learning). Similarly, the example visualization 500B shows a pie graph of calm and stress levels experienced by the user 102 during meetings when the user's 102 spouse works from home. The example visualizations 500C, 500D show bar graphs of stress and calm levels based upon whether the user's 102 kids school from home and whether the user's 102 spouse works from home, respectively. The example visualizations 500A-500D can be manipulated using filters 502.

The example visualizations 300, 400A-400B, 500A-500D can be created by the mental health tracking application 110 as the feedback 128 or a portion thereof. Alternatively or additionally, the mental health tracking application 110 can leverage the functionality of the mental health tracking system 134, and particularly, the real-time mental health tracking application 136 and/or the historical mental health tracking application 138 to create the example visualizations 300, 400A-400B, 500A-500D. In some embodiments, the data to be visualized can be provided to a data visualization platform such as POWER BI available from MICROSOFT CORPORATION

Turning now to FIG. 6, a block diagram illustrating a computer system 600 configured to provide the functionality described herein in accordance with various embodiments of the concepts and technologies disclosed herein. In some embodiments, the user system 108, the calendar system 122, the other system(s) 126, and/or the mental health tracking system 134 can be configured the same as or similar to the computer system 600. The computer system 600 includes a processing unit 602, a memory 604, one or more user interface devices 606, one or more input/output (“I/O”) devices 608, and one or more network devices 610, each of which is operatively connected to a system bus 612. The bus 612 enables bi-directional communication between the processing unit 602, the memory 604, the user interface devices 606, the I/O devices 608, and the network devices 610.

The processing unit 602 may be a standard central processor that performs arithmetic and logical operations, a more specific purpose programmable logic controller (“PLC”), a programmable gate array, or other type of processor known to those skilled in the art and suitable for controlling the operation of the server computer. The processing unit 602 can be a single processing unit or a multiple processing unit that includes more than one processing component. Processing units are generally known, and therefore are not described in further detail herein.

The memory 604 communicates with the processing unit 602 via the system bus 612. The memory 604 can include a single memory component or multiple memory components. In some embodiments, the memory 604 is operatively connected to a memory controller (not shown) that enables communication with the processing unit 602 via the system bus 612. The memory 604 includes an operating system 614 and one or more program modules 616. The operating system 614 can include, but is not limited to, members of the WINDOWS, WINDOWS CE, and/or WINDOWS MOBILE families of operating systems from MICROSOFT CORPORATION, the LINUX family of operating systems, the SYMBIAN family of operating systems from SYMBIAN LIMITED, the BREW family of operating systems from QUALCOMM CORPORATION, the MAC OS, iOS, and/or LEOPARD families of operating systems from APPLE CORPORATION, the FREEBSD family of operating systems, the SOLARIS family of operating systems from ORACLE CORPORATION, other operating systems, and the like.

The program modules 616 may include various software and/or program modules described herein. In some embodiments, the program modules 616 in the user system 108 configured like the computer system 600 can include, for example, the mental health tracking application 110, the calendar application 112, the other application(s) 114, or a combination thereof. In some embodiments, the program modules 616 in the mental health tracking system 132 configured like the computer system 600 can include, for example, the real-time mental health tracking application 136, the historical mental health tracking application 138, or a combination thereof. In some embodiments, multiple implementations of the computer system 600 can be used, wherein each implementation is configured to execute one or more of the program modules 616. The program modules 616 and/or other programs can be embodied in computer-readable media containing instructions that, when executed by the processing unit 602, perform the methods described herein. According to embodiments, the program modules 616 may be embodied in hardware, software, firmware, or any combination thereof. Although not shown in FIG. 6, it should be understood that the memory 604 also can be configured to store the EEG data 106, the calendar data 116, the other data 124, the machine learning model(s) 128, the feedback 128, combinations thereof, and/or other data disclosed herein.

By way of example, and not limitation, computer-readable media may include any available computer storage media or communication media that can be accessed by the computer system 600. Communication media includes computer-readable instructions, data structures, program modules, or other data in a modulated data signal such as a carrier wave or other transport mechanism and includes any delivery media. The term “modulated data signal” means a signal that has one or more of its characteristics changed or set in a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

Computer storage media includes volatile and non-volatile, removable and non-removable media implemented in any method or technology for storage of information such as computer-readable instructions, data structures, program modules, or other data. Computer storage media includes, but is not limited to, RAM, ROM, Erasable Programmable ROM (“EPROM”), Electrically Erasable Programmable ROM (“EEPROM”), flash memory or other solid state memory technology, CD-ROM, digital versatile disks (“DVD”), or other optical storage, magnetic cassettes, magnetic tape, magnetic disk storage or other magnetic storage devices, or any other medium which can be used to store the desired information and which can be accessed by the computer system 600. In the claims, the phrase “computer storage medium,” “computer-readable storage medium,” and variations thereof does not include waves or signals per se and/or communication media, and therefore should be construed as being directed to “non-transitory” media only.

The user interface devices 606 may include one or more devices with which a user accesses the computer system 600. The user interface devices 606 may include, but are not limited to, computers, servers, personal digital assistants, cellular phones, or any suitable computing devices. The I/O devices 608 enable a user to interface with the program modules 616. In one embodiment, the I/O devices 608 are operatively connected to an I/O controller (not shown) that enables communication with the processing unit 602 via the system bus 612. The I/O devices 608 may include one or more input devices, such as, but not limited to, a keyboard, a mouse, or an electronic stylus. Further, the I/O devices 608 may include one or more output devices, such as, but not limited to, a display screen or a printer.

The network devices 610 enable the computer system 600 to communicate with other networks or remote systems via the network 132. Examples of the network devices 610 include, but are not limited to, a modem, a radio frequency (“RF”) or infrared (“IR”) transceiver, a telephonic interface, a bridge, a router, or a network card. The network 132 may include a wireless network such as, but not limited to, a Wireless Local Area Network (“WLAN”) such as a WI-FI network, a Wireless Wide Area Network (“WWAN”), a Wireless Personal Area Network (“WPAN”) such as BLUETOOTH, a Wireless Metropolitan Area Network (“WMAN”) such a WiMAX network, or a cellular network. Alternatively, the network 132 may be a wired network such as, but not limited to, a Wide Area Network (“WAN”) such as the Internet, a Local Area Network (“LAN”) such as the Ethernet, a wired Personal Area Network (“PAN”), or a wired Metropolitan Area Network (“MAN”).

Turning now to FIG. 7, an illustrative mobile device 700 and components thereof will be described. In some embodiments, the user system 108 described herein can be configured similar to or the same as the mobile device 700. While connections are not shown between the various components illustrated in FIG. 7, it should be understood that some, none, or all of the components illustrated in FIG. 7 can be configured to interact with one another to carry out various device functions. In some embodiments, the components are arranged so as to communicate via one or more busses (not shown). Thus, it should be understood that FIG. 7 and the following description are intended to provide a general understanding of a suitable environment in which various aspects of embodiments can be implemented, and should not be construed as being limiting in any way.

As illustrated in FIG. 7, the mobile device 700 can include a display 702 for displaying data. According to various embodiments, the display 702 can be configured to display various GUI elements, text, images, video, virtual keypads and/or keyboards, messaging data, notification messages, metadata, Internet content, device status, time, date, calendar data, device preferences, map and location data, combinations thereof, and/or the like. The mobile device 700 also can include a processor 704 and a memory or other data storage device (“memory”) 706. The processor 704 can be configured to process data and/or can execute computer-executable instructions stored in the memory 706. The computer-executable instructions executed by the processor 704 can include, for example, an operating system 708, one or more applications 710, other computer-executable instructions stored in the memory 706, or the like. In some embodiments, the applications 710 also can include a UI application (not illustrated in FIG. 7).

The UI application can interface with the operating system 708 to facilitate user interaction with functionality and/or data stored at the mobile device 700 and/or stored elsewhere. In some embodiments, the operating system 708 can include a member of the SYMBIAN OS family of operating systems from SYMBIAN LIMITED, a member of the WINDOWS MOBILE OS and/or WINDOWS PHONE OS families of operating systems from MICROSOFT CORPORATION, a member of the PALM WEBOS family of operating systems from HEWLETT PACKARD CORPORATION, a member of the BLACKBERRY OS family of operating systems from RESEARCH IN MOTION LIMITED, a member of the IOS family of operating systems from APPLE INC., a member of the ANDROID OS family of operating systems from GOOGLE INC., and/or other operating systems. These operating systems are merely illustrative of some contemplated operating systems that may be used in accordance with various embodiments of the concepts and technologies described herein and therefore should not be construed as being limiting in any way.

The UI application can be executed by the processor 704 to aid a user in entering/deleting data, entering and setting user IDs and passwords for device access, configuring settings, manipulating content and/or settings, multimode interaction, interacting with other applications 710, and otherwise facilitating user interaction with the operating system 708, the applications 710, and/or other types or instances of data 712 that can be stored at the mobile device 700.

The applications 710, the data 712, and/or portions thereof can be stored in the memory 706 and/or in a firmware 714, and can be executed by the processor 704. The applications 710 can include the mental health tracking application 110, the calendar application 112, the other application(s) 114, or some combination thereof. The data 712 can include the EEG data 106, the calendar data 116, the other data 124, the feedback 128, the machine learning models 130, or some combination thereof.

The firmware 714 also can store code for execution during device power up and power down operations. It can be appreciated that the firmware 714 can be stored in a volatile or non-volatile data storage device including, but not limited to, the memory 706 and/or a portion thereof.

The mobile device 700 also can include an input/output (“I/O”) interface 716. The I/O interface 716 can be configured to support the input/output of data such as location information, presence status information, user IDs, passwords, and application initiation (start-up) requests. In some embodiments, the I/O interface 716 can include a hardwire connection such as a universal serial bus (“USB”) port, a mini-USB port, a micro-USB port, an audio jack, a PS2 port, an IEEE 1394 (“FIREWIRE”) port, a serial port, a parallel port, an Ethernet (RJ45) port, an RJ11 port, a proprietary port, combinations thereof, or the like. In some embodiments, the mobile device 700 can be configured to synchronize with another device to transfer content to and/or from the mobile device 700. In some embodiments, the mobile device 700 can be configured to receive updates to one or more of the applications 710 via the I/O interface 716, though this is not necessarily the case. In some embodiments, the I/O interface 716 accepts I/O devices such as keyboards, keypads, mice, interface tethers, printers, plotters, external storage, touch/multi-touch screens, touch pads, trackballs, joysticks, microphones, remote control devices, displays, projectors, medical equipment (e.g., stethoscopes, heart monitors, and other health metric monitors), modems, routers, external power sources, docking stations, combinations thereof, and the like. It should be appreciated that the I/O interface 716 may be used for communications between the mobile device 700 and a network device or local device.

The mobile device 700 also can include a communications component 718. The communications component 718 can be configured to interface with the processor 704 to facilitate wired and/or wireless communications with one or more networks, such as the network 132, the Internet, or some combination thereof. In some embodiments, the communications component 718 includes a multimode communications subsystem for facilitating communications via the cellular network and one or more other networks.

The communications component 718, in some embodiments, includes one or more transceivers. The one or more transceivers, if included, can be configured to communicate over the same and/or different wireless technology standards with respect to one another. For example, in some embodiments, one or more of the transceivers of the communications component 718 may be configured to communicate using Global System for Mobile communications (“GSM”), Code-Division Multiple Access (“CDMA”) CDMAONE, CDMA2000, Long-Term Evolution (“LTE”) LTE, and various other 2G, 2.5G, 3G, 4G, 4.5G, 5G, and greater generation technology standards. Moreover, the communications component 718 may facilitate communications over various channel access methods (which may or may not be used by the aforementioned standards) including, but not limited to, Time-Division Multiple Access (“TDMA”), Frequency-Division Multiple Access (“FDMA”), Wideband CDMA (“W-CDMA”), Orthogonal Frequency-Division Multiple Access (“OFDMA”), Space-Division Multiple Access (“SDMA”), and the like.

In addition, the communications component 718 may facilitate data communications using General Packet Radio Service (“GPRS”), Enhanced Data services for Global Evolution (“EDGE”), the High-Speed Packet Access (“HSPA”) protocol family including High-Speed Downlink Packet Access (“HSDPA”), Enhanced Uplink (“EUL”) (also referred to as High-Speed Uplink Packet Access (“HSUPA”), HSPA+, and various other current and future wireless data access standards. In the illustrated embodiment, the communications component 718 can include a first transceiver (“TxRx”) 720A that can operate in a first communications mode (e.g., GSM). The communications component 718 also can include an N^{th} transceiver (“TxRx”) 720N that can operate in a second communications mode relative to the first transceiver 720A (e.g., UMTS). While two transceivers 720A-720N (hereinafter collectively and/or generically referred to as “transceivers 720”) are shown in FIG. 7, it should be appreciated that less than two, two, and/or more than two transceivers 720 can be included in the communications component 718.

The communications component 718 also can include an alternative transceiver (“Alt TxRx”) 722 for supporting other types and/or standards of communications. According to various contemplated embodiments, the alternative transceiver 722 can communicate using various communications technologies such as, for example, WI-FI, WIMAX, BLUETOOTH, infrared, infrared data association (“IRDA”), near field communications (“NFC”), other RF technologies, combinations thereof, and the like. In some embodiments, the communications component 718 also can facilitate reception from terrestrial radio networks, digital satellite radio networks, internet-based radio service networks, combinations thereof, and the like. The communications component 718 can process data from a network such as the Internet, an intranet, a broadband network, a WI-FI hotspot, an Internet service provider (“ISP”), a digital subscriber line (“DSL”) provider, a broadband provider, combinations thereof, or the like.

The mobile device 700 also can include one or more sensors 724. The sensors 724 can include temperature sensors, light sensors, air quality sensors, movement sensors, accelerometers, magnetometers, gyroscopes, infrared sensors, orientation sensors, noise sensors, microphones proximity sensors, combinations thereof, and/or the like. Additionally, audio capabilities for the mobile device 700 may be provided by an audio I/O component 726. The audio I/O component 726 of the mobile device 700 can include one or more speakers for the output of audio signals, one or more microphones for the collection and/or input of audio signals, and/or other audio input and/or output devices.

The illustrated mobile device 700 also can include a subscriber identity module (“SIM”) system 728. The SIM system 728 can include a universal SIM (“USIM”), a universal integrated circuit card (“UICC”) and/or other identity devices. The SIM system 728 can include and/or can be connected to or inserted into an interface such as a slot interface 730. In some embodiments, the slot interface 730 can be configured to accept insertion of other identity cards or modules for accessing various types of networks. Additionally, or alternatively, the slot interface 730 can be configured to accept multiple subscriber identity cards. Because other devices and/or modules for identifying users and/or the mobile device 700 are contemplated, it should be understood that these embodiments are illustrative, and should not be construed as being limiting in any way.

The mobile device 700 also can include an image capture and processing system 732 (“image system”). The image system 732 can be configured to capture or otherwise obtain photos, videos, and/or other visual information. As such, the image system 732 can include cameras, lenses, charge-coupled devices (“CCDs”), combinations thereof, or the like. The mobile device 700 may also include a video system 734. The video system 736 can be configured to capture, process, record, modify, and/or store video content. Photos and videos obtained using the image system 732 and the video system 734, respectively, may be added as message content to an MMS message, email message, and sent to another device. The video and/or photo content also can be shared with other devices via various types of data transfers via wired and/or wireless communication devices as described herein.

The mobile device 700 also can include one or more location components 736. The location components 736 can be configured to send and/or receive signals to determine a geographic location of the mobile device 700. According to various embodiments, the location components 736 can send and/or receive signals from global positioning system (“GPS”) devices, assisted-GPS (“A-GPS”) devices, WI-FI/WIMAX and/or cellular network triangulation data, combinations thereof, and the like. The location component 736 also can be configured to communicate with the communications component 718 to retrieve triangulation data for determining a location of the mobile device 700. In some embodiments, the location component 736 can interface with cellular network nodes, telephone lines, satellites, location transmitters and/or beacons, wireless network transmitters and receivers, combinations thereof, and the like. In some embodiments, the location component 736 can include and/or can communicate with one or more of the sensors 724 such as a compass, an accelerometer, and/or a gyroscope to determine the orientation of the mobile device 700. Using the location component 736, the mobile device 700 can generate and/or receive data to identify its geographic location, or to transmit data used by other devices to determine the location of the mobile device 700. The location component 736 may include multiple components for determining the location and/or orientation of the mobile device 700.

The illustrated mobile device 700 also can include a power source 738. The power source 738 can include one or more batteries, power supplies, power cells, and/or other power subsystems including alternating current (“AC”) and/or direct current (“DC”) power devices. The power source 738 also can interface with an external power system or charging equipment via a power I/O component 740. Because the mobile device 700 can include additional and/or alternative components, the above embodiment should be understood as being illustrative of one possible operating environment for various embodiments of the concepts and technologies described herein. The described embodiment of the mobile device 700 is illustrative, and should not be construed as being limiting in any way.

As used herein, communication media includes computer-executable instructions, data structures, program modules, or other data in a modulated data signal such as a carrier wave or other transport mechanism and includes any delivery media. The term “modulated data signal” means a signal that has one or more of its characteristics changed or set in a manner as to encode information in the signal. By way of example, and not limitation, communication media includes wired media such as a wired network or direct-wired connection, and wireless media such as acoustic, RF, infrared, and other wireless media. Combinations of the any of the above should also be included within the scope of computer-readable media.

By way of example, and not limitation, computer storage media may include volatile and non-volatile, removable and non-removable media implemented in any method or technology for storage of information such as computer-executable instructions, data structures, program modules, or other data. For example, computer media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the mobile device 700 or other devices or computers described herein. In the claims, the phrase “computer storage medium,” “computer-readable storage medium,” and variations thereof does not include waves or signals per se and/or communication media, and therefore should be construed as being directed to “non-transitory” media only.

Encoding the software modules presented herein also may transform the physical structure of the computer-readable media presented herein. The specific transformation of physical structure may depend on various factors, in different implementations of this description. Examples of such factors may include, but are not limited to, the technology used to implement the computer-readable media, whether the computer-readable media is characterized as primary or secondary storage, and the like. For example, if the computer-readable media is implemented as semiconductor-based memory, the software disclosed herein may be encoded on the computer-readable media by transforming the physical state of the semiconductor memory. For example, the software may transform the state of transistors, capacitors, or other discrete circuit elements constituting the semiconductor memory. The software also may transform the physical state of such components in order to store data thereupon.

As another example, the computer-readable media disclosed herein may be implemented using magnetic or optical technology. In such implementations, the software presented herein may transform the physical state of magnetic or optical media, when the software is encoded therein. These transformations may include altering the magnetic characteristics of particular locations within given magnetic media. These transformations also may include altering the physical features or characteristics of particular locations within given optical media, to change the optical characteristics of those locations. Other transformations of physical media are possible without departing from the scope and spirit of the present description, with the foregoing examples provided only to facilitate this discussion.

In light of the above, it should be appreciated that many types of physical transformations may take place in the mobile device 700 in order to store and execute the software components presented herein. It is also contemplated that the mobile device 700 may not include all of the components shown in FIG. 7, may include other components that are not explicitly shown in FIG. 7, or may utilize an architecture completely different than that shown in FIG. 7.

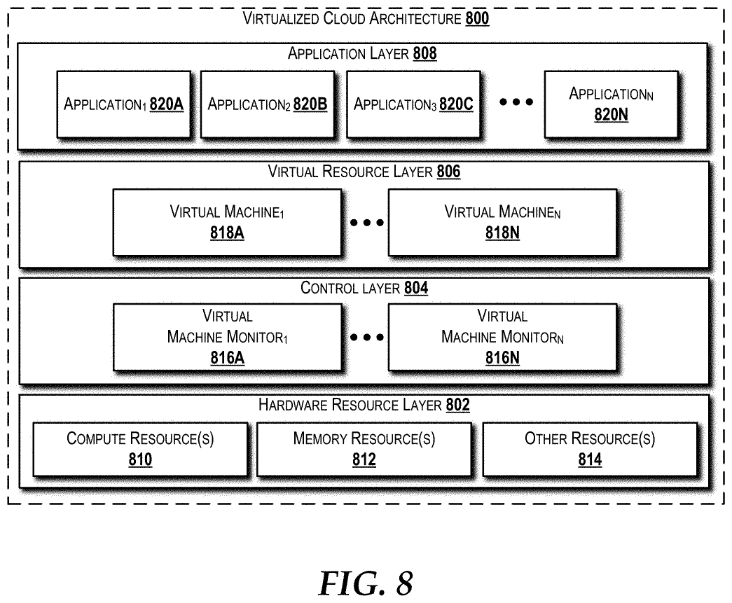

Turning now to FIG. 8, a block diagram illustrating an example virtualized cloud architecture 800 and components thereof will be described, according to an exemplary embodiment. The virtualized cloud architecture 800 can be utilized to implement various elements disclosed herein. In some embodiments, the mental health tracking system 134, at least in part, is implemented in the virtualized cloud architecture 800.

The virtualized cloud architecture 800 is a shared infrastructure that can support multiple services and network applications. The illustrated virtualized cloud architecture 800 includes a hardware resource layer 802, a control layer 804, a virtual resource layer 806, and an application layer 808 that work together to perform operations as will be described in detail herein.

The hardware resource layer 802 provides hardware resources, which, in the illustrated embodiment, include one or more compute resources 810, one or more memory resources 812, and one or more other resources 814. The compute resource(s) 810 can include one or more hardware components that perform computations to process data, and/or to execute computer-executable instructions of one or more application programs, operating systems, and/or other software. The compute resources 810 can include one or more central processing units (“CPUs”) configured with one or more processing cores. The compute resources 810 can include one or more graphics processing unit (“GPU”) configured to accelerate operations performed by one or more CPUs, and/or to perform computations to process data, and/or to execute computer-executable instructions of one or more application programs, operating systems, and/or other software that may or may not include instructions particular to graphics computations. In some embodiments, the compute resources 810 can include one or more discrete GPUs. In some other embodiments, the compute resources 810 can include CPU and GPU components that are configured in accordance with a co-processing CPU/GPU computing model, wherein the sequential part of an application executes on the CPU and the computationally-intensive part is accelerated by the GPU. The compute resources 810 can include one or more system-on-chip (“SoC”) components along with one or more other components, including, for example, one or more of the memory resources 812, and/or one or more of the other resources 814. In some embodiments, the compute resources 810 can be or can include one or more SNAPDRAGON SoCs, available from QUALCOMM; one or more TEGRA SoCs, available from NVIDIA; one or more HUMMINGBIRD SoCs, available from SAMSUNG; one or more Open Multimedia Application Platform (“OMAP”) SoCs, available from TEXAS INSTRUMENTS; one or more customized versions of any of the above SoCs; and/or one or more proprietary SoCs. The compute resources 810 can be or can include one or more hardware components architected in accordance with an advanced reduced instruction set computing (“RISC”) machine (“ARM”) architecture, available for license from ARM HOLDINGS. Alternatively, the compute resources 810 can be or can include one or more hardware components architected in accordance with an x86 architecture, such an architecture available from INTEL CORPORATION of Mountain View, Calif., and others. Those skilled in the art will appreciate the implementation of the compute resources 810 can utilize various computation architectures, and as such, the compute resources 810 should not be construed as being limited to any particular computation architecture or combination of computation architectures, including those explicitly disclosed herein.

The memory resource(s) 812 can include one or more hardware components that perform storage operations, including temporary or permanent storage operations. In some embodiments, the memory resource(s) 812 include volatile and/or non-volatile memory implemented in any method or technology for storage of information such as computer-readable instructions, data structures, program modules, or other data disclosed herein. Computer storage media includes, but is not limited to, random access memory (“RAM”), read-only memory (“ROM”), Erasable Programmable ROM (“EPROM”), Electrically Erasable Programmable ROM (“EEPROM”), flash memory or other solid state memory technology, CD-ROM, digital versatile disks (“DVD”), or other optical storage, magnetic cassettes, magnetic tape, magnetic disk storage or other magnetic storage devices, or any other medium which can be used to store data and which can be accessed by the compute resources 810.

The other resource(s) 814 can include any other hardware resources that can be utilized by the compute resources(s) 810 and/or the memory resource(s) 812 to perform operations described herein. The other resource(s) 814 can include one or more input and/or output processors (e.g., network interface controller or wireless radio), one or more modems, one or more codec chipset, one or more pipeline processors, one or more fast Fourier transform (“FFT”) processors, one or more digital signal processors (“DSPs”), one or more speech synthesizers, and/or the like.

The hardware resources operating within the hardware resource layer 802 can be virtualized by one or more virtual machine monitors (“VMMs”) 816A-816N (also known as “hypervisors”; hereinafter “VMMs 816”) operating within the control layer 804 to manage one or more virtual resources that reside in the virtual resource layer 806. The VMMs 816 can be or can include software, firmware, and/or hardware that alone or in combination with other software, firmware, and/or hardware, manages one or more virtual resources operating within the virtual resource layer 806.

The virtual resources operating within the virtual resource layer 806 can include abstractions of at least a portion of the compute resources 810, the memory resources 812, the other resources 814, or any combination thereof. These abstractions are referred to herein as virtual machines (“VMs”). In the illustrated embodiment, the virtual resource layer 806 includes VMs 818A-818N (hereinafter “VMs 818”). Each of the VMs 818 can execute one or more applications 820A-820N in the application layer 808. The applications 820A-820N can include the real-time mental health tracking application 136 and the historical mental health tracking application 138.

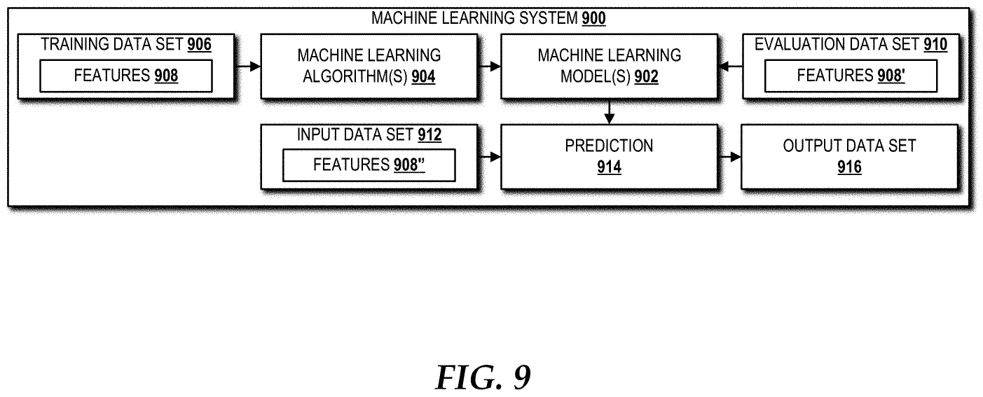

Turning now to FIG. 9, a machine learning system 900 capable of implementing aspects of the embodiments disclosed herein will be described. In some embodiments, aspects of the user system 108 and/or the mental health tracking system 134 can use machine learning and/or artificial intelligence applications. Accordingly, the user system 108 and/or the mental health tracking system 132 can include the machine learning system 900 or can be in communication with the machine learning system 900.

The illustrated machine learning system 900 includes one or more machine learning models 902, such as the machine learning models 130 mentioned above. The machine learning models 902 can include supervised and/or semi-supervised learning models. The machine learning model(s) 902 can be created by the machine learning system 900 based upon one or more machine learning algorithms 904. The machine learning algorithm(s) 904 can be any existing, well-known algorithm, any proprietary algorithms, or any future machine learning algorithm. Some example machine learning algorithms 904 include, but are not limited to, neural networks, gradient descent, linear regression, logistic regression, linear discriminant analysis, classification tree, regression tree, Naive Bayes, K-nearest neighbor, learning vector quantization, support vector machines, and the like. Classification and regression algorithms might find particular applicability to the concepts and technologies disclosed herein. Those skilled in the art will appreciate the applicability of various machine learning algorithms 904 based upon the problem(s) to be solved by machine learning via the machine learning system 900.

The machine learning system 900 can control the creation of the machine learning models 902 via one or more training parameters. In some embodiments, the training parameters are selected modelers at the direction of an enterprise, for example. Alternatively, in some embodiments, the training parameters are automatically selected based upon data provided in one or more training data sets 906. The training parameters can include, for example, a learning rate, a model size, a number of training passes, data shuffling, regularization, and/or other training parameters known to those skilled in the art. The training data in the training data sets 906.

The learning rate is a training parameter defined by a constant value. The learning rate affects the speed at which the machine learning algorithm 904 converges to the optimal weights. The machine learning algorithm 904 can update the weights for every data example included in the training data set 906. The size of an update is controlled by the learning rate. A learning rate that is too high might prevent the machine learning algorithm 904 from converging to the optimal weights. A learning rate that is too low might result in the machine learning algorithm 904 requiring multiple training passes to converge to the optimal weights.

The model size is regulated by the number of input features (“features”) 908 in the training data set 906. A greater the number of features 908 yields a greater number of possible patterns that can be determined from the training data set 906. The model size should be selected to balance the resources (e.g., compute, memory, storage, etc.) needed for training and the predictive power of the resultant machine learning model 902.

The number of training passes indicates the number of training passes that the machine learning algorithm 904 makes over the training data set 906 during the training process. The number of training passes can be adjusted based, for example, on the size of the training data set 906, with larger training data sets being exposed to fewer training passes in consideration of time and/or resource utilization. The effectiveness of the resultant machine learning model 902 can be increased by multiple training passes.

Data shuffling is a training parameter designed to prevent the machine learning algorithm 904 from reaching false optimal weights due to the order in which data contained in the training data set 906 is processed. For example, data provided in rows and columns might be analyzed first row, second row, third row, etc., and thus an optimal weight might be obtained well before a full range of data has been considered. By data shuffling, the data contained in the training data set 906 can be analyzed more thoroughly and mitigate bias in the resultant machine learning model 902.

Regularization is a training parameter that helps to prevent the machine learning model 902 from memorizing training data from the training data set 906. In other words, the machine learning model 902 fits the training data set 906, but the predictive performance of the machine learning model 902 is not acceptable. Regularization helps the machine learning system 900 avoid this overfitting/memorization problem by adjusting extreme weight values of the features 908. For example, a feature that has a small weight value relative to the weight values of the other features in the training data set 906 can be adjusted to zero.

The machine learning system 900 can determine model accuracy after training by using one or more evaluation data sets 910 containing the same features 908′ as the features 908 in the training data set 906. This also prevents the machine learning model 902 from simply memorizing the data contained in the training data set 906. The number of evaluation passes made by the machine learning system 900 can be regulated by a target model accuracy that, when reached, ends the evaluation process and the machine learning model 902 is considered ready for deployment.

After deployment, the machine learning model 902 can perform a prediction operation (“prediction”) 914 with an input data set 912 having the same features 908″ as the features 908 in the training data set 906 and the features 908′ of the evaluation data set 910. The results of the prediction 914 are included in an output data set 916 consisting of predicted data. The machine learning model 902 can perform other operations, such as regression, classification, and others. As such, the example illustrated in FIG. 9 should not be construed as being limiting in any way.

Turning now to FIG. 10, details of the network 132 are illustrated, according to an illustrative embodiment. The network 132 includes a cellular network 1002, a packet data network 1004, and a circuit switched network 1006. The cellular network 1002 can include various components such as, but not limited to, base transceiver stations (“BTSs”), Node-Bs or e-Node-Bs, base station controllers (“BSCs”), radio network controllers (“RNCs”), mobile switching centers (“MSCs”), mobility management entities (“MMEs”), short message service centers (“SMSCs”), multimedia messaging service centers (“MMSCs”), home location registers (“HLRs”), home subscriber servers (“HSSs”), visitor location registers (“VLRs”), charging platforms, billing platforms, voicemail platforms, GPRS core network components, location service nodes, and the like. The cellular network 1002 also includes radios and nodes for receiving and transmitting voice, data, and combinations thereof to and from radio transceivers, networks, the packet data network 1004, and the circuit switched network 1006.

A mobile communications device 1008, such as, for example, a cellular telephone, a user equipment, a mobile terminal, a PDA, a laptop computer, a handheld computer, and combinations thereof, can be operatively connected to the cellular network 1002. The cellular network 1002 can be configured as a GSM) network and can provide data communications via GPRS and/or EDGE. Additionally, or alternatively, the cellular network 1002 can be configured as a 3G Universal Mobile Telecommunications System (“UMTS”) network and can provide data communications via the HSPA protocol family, for example, HSDPA, EUL, and HSPA+. The cellular network 1002 also is compatible with 4G mobile communications standards such as LTE, 5G mobile communications standards, or the like, as well as evolved and future mobile standards.

The packet data network 1004 includes various systems, devices, servers, computers, databases, and other devices in communication with one another, as is generally known. In some embodiments, the packet data network 1004 is or includes one or more WI-FI networks, each of which can include one or more WI-FI access points, routers, switches, and other WI-FI network components. The packet data network 1004 devices are accessible via one or more network links. The servers often store various files that are provided to a requesting device such as, for example, a computer, a terminal, a smartphone, or the like. Typically, the requesting device includes software for executing a web page in a format readable by the browser or other software. Other files and/or data may be accessible via “links” in the retrieved files, as is generally known. In some embodiments, the packet data network 1004 includes or is in communication with the Internet. The circuit switched network 1006 includes various hardware and software for providing circuit switched communications. The circuit switched network 1006 may include, or may be, what is often referred to as a plain old telephone system (“POTS”). The functionality of a circuit switched network 1006 or other circuit-switched network are generally known and will not be described herein in detail.

The illustrated cellular network 1002 is shown in communication with the packet data network 1004 and a circuit switched network 1006, though it should be appreciated that this is not necessarily the case. One or more Internet-capable devices 1010 such as a laptop, a portable device, or another suitable device, can communicate with one or more cellular networks 1002, and devices connected thereto, through the packet data network 1004. It also should be appreciated that the Internet-capable device 1010 can communicate with the packet data network 1004 through the circuit switched network 1006, the cellular network 1002, and/or via other networks (not illustrated).

As illustrated, a communications device 1012, for example, a telephone, facsimile machine, modem, computer, or the like, can be in communication with the circuit switched network 1006, and therethrough to the packet data network 1004 and/or the cellular network 1002. It should be appreciated that the communications device 1012 can be an Internet-capable device, and can be substantially similar to the Internet-capable device 1010.

Based on the foregoing, it should be appreciated that concepts and technologies directed to mental health tracking have been disclosed herein. Although the subject matter presented herein has been described in language specific to computer structural features, methodological and transformative acts, specific computing machinery, and computer-readable media, it is to be understood that the concepts and technologies disclosed herein are not necessarily limited to the specific features, acts, or media described herein. Rather, the specific features, acts and mediums are disclosed as example forms of implementing the concepts and technologies disclosed herein.

The subject matter described above is provided by way of illustration only and should not be construed as limiting. Various modifications and changes may be made to the subject matter described herein without following the example embodiments and applications illustrated and described, and without departing from the true spirit and scope of the embodiments of the concepts and technologies disclosed herein.

1. A method comprising:
obtaining, by a system comprising a processor, electroencephalography data associated with a user;
obtaining, by the system, calendar data associated with the user, wherein the calendar data identifies a calendar event;
mapping, by the system, the calendar data to the electroencephalography data; and
providing, by the system, feedback to the user, wherein the feedback provides insight regarding a stress level of the user in association with the calendar event.

2. The method of claim 1, wherein obtaining, by the system, the electroencephalography data associated with the user comprises obtaining, by the system, the electroencephalography data from an electroencephalography device.

3. The method of claim 2, wherein the electroencephalography data comprises real-time electroencephalography data.

4. The method of claim 2, wherein the electroencephalography data comprises historical electroencephalography data.

5. The method of claim 1, wherein the calendar data is associated with a calendar of the user; and wherein the user is a participant in the calendar event.

6. The method of claim 1, wherein the calendar data is associated with a shared calendar of an entity other than the user.

7. The method of claim 1, further comprising obtaining, by the system, other data associated with the user; and wherein mapping, by the system, the calendar data to the electroencephalography data comprises mapping, by the system, the calendar data and the other data to the electroencephalography data.

8. The method of claim 1, wherein the feedback comprises a visualization of the stress level of the user in association with the calendar event.

9. A system comprising:
a processor;
a memory that stores instructions that, when executed by the processor, cause the processor to perform operations comprising
obtaining electroencephalography data associated with a user,
obtaining calendar data associated with the user, wherein the calendar data identifies a calendar event,
mapping the calendar data to the electroencephalography data, and
providing feedback to the user, wherein the feedback provides insight regarding a stress level of the user in association with the calendar event.

10. The system of claim 9, wherein obtaining the electroencephalography data associated with the user comprises obtaining, by the system, the electroencephalography data from an electroencephalography device.

11. The system of claim 10, wherein the electroencephalography data comprises real-time electroencephalography data or historical electroencephalography data.

12. The system of claim 9, wherein the calendar data is associated with a calendar of the user; and wherein the user is a participant in the calendar event.

13. The system of claim 9, wherein the calendar data is associated with a shared calendar of an entity other than the user.

14. The system of claim 9, wherein the operations further comprise obtaining other data associated with the user; and wherein mapping the calendar data to the electroencephalography data comprises mapping the calendar data and the other data to the electroencephalography data.

15. The system of claim 9, wherein the feedback comprises a visualization of the stress level of the user in association with the calendar event.

16. A computer-readable storage medium having instructions stored thereon that, when executed by a processor of a system, cause the processor to perform operations comprising:
obtaining electroencephalography data associated with a user;
obtaining calendar data associated with the user, wherein the calendar data identifies a calendar event;
mapping the calendar data to the electroencephalography data; and
providing feedback to the user, wherein the feedback provides insight regarding a stress level of the user in association with the calendar event.

17. The computer-readable storage medium of claim 16, wherein obtaining the electroencephalography data associated with the user comprises obtaining, by the system, the electroencephalography data from an electroencephalography device.

18. The computer-readable storage medium of claim 17, wherein the electroencephalography data comprises real-time electroencephalography data or historical electroencephalography data.

19. The computer-readable storage medium of claim 16, wherein the operations further comprise obtaining other data associated with the user; and wherein mapping the calendar data to the electroencephalography data comprises mapping the calendar data and the other data to the electroencephalography data.

20. The computer-readable storage medium of claim 16, wherein the feedback comprises a visualization of the stress level of the user in association with the calendar event.
